(19) United States
(12) Patent Application Publication
(10) Pub. No.: US 2022/0313791 A1
(43) Pub. Date: Oct. 6, 2022

(54) Composition for Collagen-Based Drink Mix

(71) Applicant: Chasity Benoit, Wichita, KS (US)

(72) Inventor: Chasity Benoit, Wichita, KS (US)

(21) Appl. No.: 17/684,616

(22) Filed: Mar. 2, 2022

Related U.S. Application Data
(60) Provisional application No. 63/168,136, filed on Mar. 30, 2021.

(51) Int. Cl. A61K 38/39; A23L 33/17; A23L 33/15; A23L 2/66; A23L 2/56; A23L 2/39; A61K 31/375; A61K 31/10; A61K 9/00

(52) U.S. Cl. CPC A61K 38/39; A23L 33/17; A23L 33/15; A23L 2/66; A23L 2/56; A23L 2/39; A61K 31/375; A61K 31/10; A61K 9/0095; A23V 2002/00

(57) ABSTRACT

A composition for collagen-based drink mix supplies a user's body with collagen while simultaneously improving the overall health of the user. The composition includes a quantity of methylsulfamemethane (MSM), a quantity of ascorbic acid, and a quantity of hydrolyzed bovine collagen. The quantity of MSM, the quantity of ascorbic acid, and the quantity of hydrolyzed bovine collagen are heterogeneously mixed into a powdered mixture. The quantity of MSM, the quantity of ascorbic acid, and the quantity of hydrolyzed bovine collagen are heterogeneously mixed into a powdered mixture. The composition further includes a quantity of flavoring that is further heterogeneously mixed into the powdered mixture.

The current application claims a priority to the U.S. provisional patent application Ser. No. 63/168,136 filed on Mar. 30, 2021.

### FIELD OF THE INVENTION

The present invention generally relates to compositions for collagen-based drink mix. More specifically, the present invention produces a drink or beverage additive that is in the form of a powder. The present invention serves to effectively supply a body with collagen while simultaneously improving the overall health of a user.

### BACKGROUND OF THE INVENTION

As people age many parts of their bodies stop acting properly as they once did when the person was younger. For example, as a person gets older their skin can deteriorate due to an individual's body not receiving the proper nutrients, resulting in dry, wrinkly and weak skin. The same type of situation can happen with joints within the body, after years of repetitive use and stressful use the joints in an individual's body can deteriorate as well. This causes limited motion for the individual as well as chronic pain in certain areas, preventing them from completing necessary daily tasks and motions. Individuals start to have issues with their body not only as they age but also after continued stressful habits and use of their body, such as extreme sports or the inability to properly deal with stress within their life. The aging and deteriorating body has continued to support a huge industry selling various products and gimmicks that claim to help fix the body or eliminate signs of aging. Many of these self-proclaimed solutions to aging or fixing the body do not properly address the root cause of the issue. For example, many beauty products and procedures aim to cover signs of aging or try to artificially fix the appearance of a person. This does not solve the problem because the signs of aging simply come back after a period of time and the person has not actually improved their health.

An objective of the present invention is to provide users with a collagen-based drink, to help eliminate damaged hair, nails, joints and provide anti-aging. The present invention intends to provide users with a formula that utilize other ingredients to boost the collagen, making its positive affects more effective. In order to accomplish that, a preferred embodiment of the present invention contains collagen, vitamin C, Methylsulfonylmethane (MSM) and a citrus flavor. Further, the citrus flavoring allows the present invention to be easily drank without a bad taste. Thus, the present invention is a collagen-based drink that utilizes vitamin C, MSM, and a citrus flavor to create a drink that improves the health of various body parts while also slowing down the aging process for the skin.

### BRIEF DESCRIPTION OF THE DRAWINGS

FIG. 1 is a block diagram of the present invention.

FIG. 2 is a block diagram illustrating the proportions of the present invention.

FIG. 3 is a block diagram of a preferred embodiment of the present invention.

FIG. 4 is a block diagram illustrating the proportions of the preferred embodiment of the present invention.

### DETAIL DESCRIPTIONS OF THE INVENTION

All illustrations of the drawings are for the purpose of describing selected versions of the present invention and are not intended to limit the scope of the present invention.

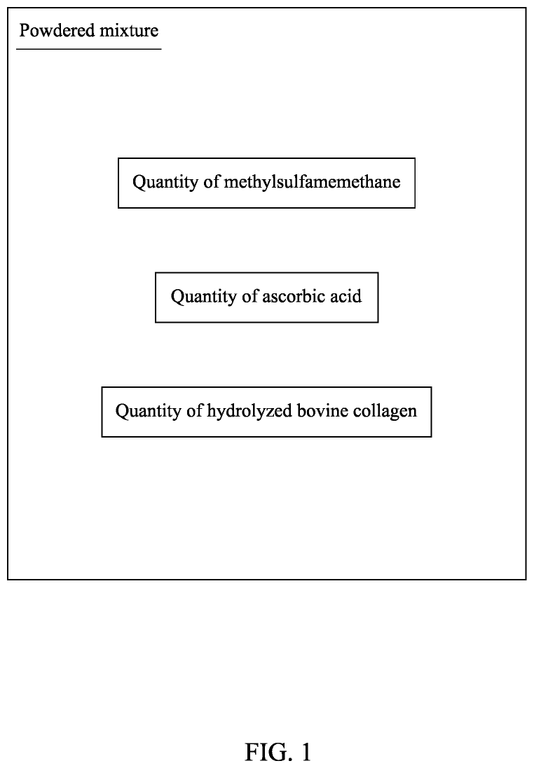

The present invention is a composition for a collagen-based drink mix that aids with the internal and the external aging of the body. The present invention provides users with an additive for a drink or beverage which boosts the effects of collagen for healthier hair, nails, joints, and so on. More specifically, the present invention helps to reduce wrinkles and dry skin, as well as improve hair quality and nail health. Moreover, the present invention helps to improve the overall immune system of a user. As seen in FIG. 1, in order to improve the health of various parts of the body simultaneously, while slowing down the aging process for the skin, the present invention comprises a quantity of methylsulfamemethane (MSM), a quantity of ascorbic acid, and a quantity of hydrolyzed bovine collagen.

The quantity of MSM enhances the effects of the quantity of hydrolyzed bovine collagen. Not only does the quantity of MSM serve to enhance the effects of the quantity of hydrolyzed bovine collagen, but the quantity of MSM features benefits that are independent of the quantity of hydrolyzed bovine collagen. The quantity of MSM significantly reduces inflammation and reduces the breakdown of cartilage, which in turn reduces joint pain and limit the damage of bones within a body. More specifically, the quantity of MSM reduces recovery time for muscles after exercise by reducing muscle damage and stress. The quantity of MSM also strengthens keratin and reduces oxidative stress which results in an external benefit of improved skin and an internal benefit of an improved immune system.

The quantity of ascorbic acid, also referred to as Vitamin C, focuses on the internal health of a body, as well as aids to boost the collagen ingredient. The quantity of ascorbic acid protects against immune system deficiencies which results in a user being healthier. Moreover, the quantity of ascorbic acid boosts the immune system to protect the user against daily viruses. The quantity of ascorbic acid aims to prevent cardiovascular disease, prenatal health issues, and a variety of eye diseases. Similar to the quantity of MSM, the quantity of ascorbic acid improves skin health.

The quantity of hydrolyzed bovine collagen targets different areas of the body simultaneously. As collagen is the most abundant protein within a body and is a major component of connective tissue, the present invention includes a high percentage of the quantity of hydrolyzed bovine collagen to provide structure to the skin, joints, hair quality, and nail health. The quantity of hydrolyzed bovine collagen improves skin health, relieves joint pain, prevents bone loss, aids in increasing muscle mass, improves heart health, and so on.

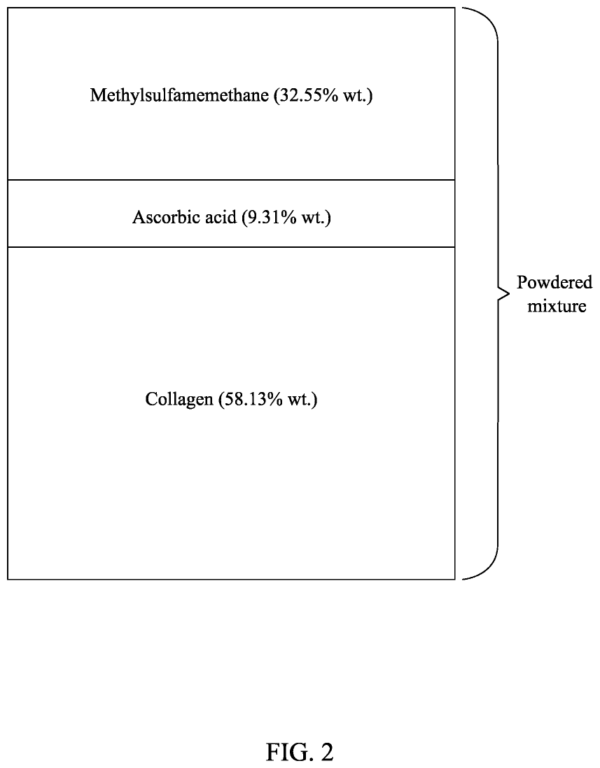

In order to receive the benefits of the combination of the aforementioned ingredients, the quantity of MSM, the quantity of ascorbic acid, and the quantity of hydrolyzed bovine collagen are heterogeneously mixed into a powdered mixture. The powdered mixture may be added to a variety of drinks and beverages to facilitate consumption. In order for the quantity of MSM and the quantity of ascorbic acid to enhance the effects of the quantity of hydrolyzed bovine collagen, the quantity of MSM is approximately 32.55 percentage by weight (wt. %) of the powdered mixture, as seen in FIG. 2. Furthermore, the quantity of ascorbic acid is approximately 9.31 wt. % of the powdered mixture. Furthermore, the quantity of hydrolyzed bovine collagen is approximately 58.13 wt. % of the powdered mixture. In reference to the aforementioned compositional proportions, the term “approximately” preferably means within an error range of up to ±0.05 wt. %.

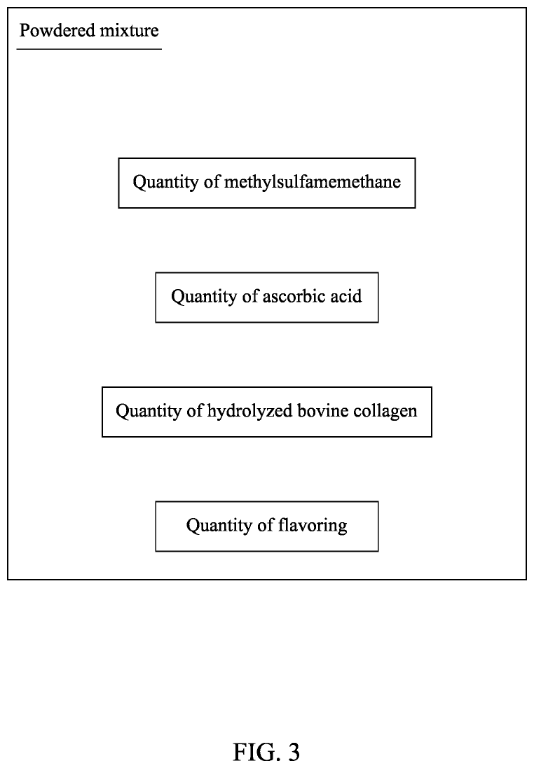

The present invention may further comprise a quantity of flavoring, as seen in FIG. 3. The quantity of flavoring provides an appealing additive to a drink or beverage. In the preferred embodiment of the present invention, the quantity of flavoring is a citrus flavor. However, it is understood that various embodiments of the present invention may comprise a quantity of flavoring of varying flavors. Moreover, the quantity of flavoring creates an appetizing taste for the present invention and allows the present invention to be more easily consumed on a regular basis. In order for a citrus flavor to be the dominant flavor of the powdered mixture, the quantity of flavoring is at least one ingredient selected from a group consisting of a quantity of citric acid, a quantity of silicon dioxide, a quantity of malic acid, a quantity of acesulfame potassium, a quantity of sucralose, and a combination thereof.

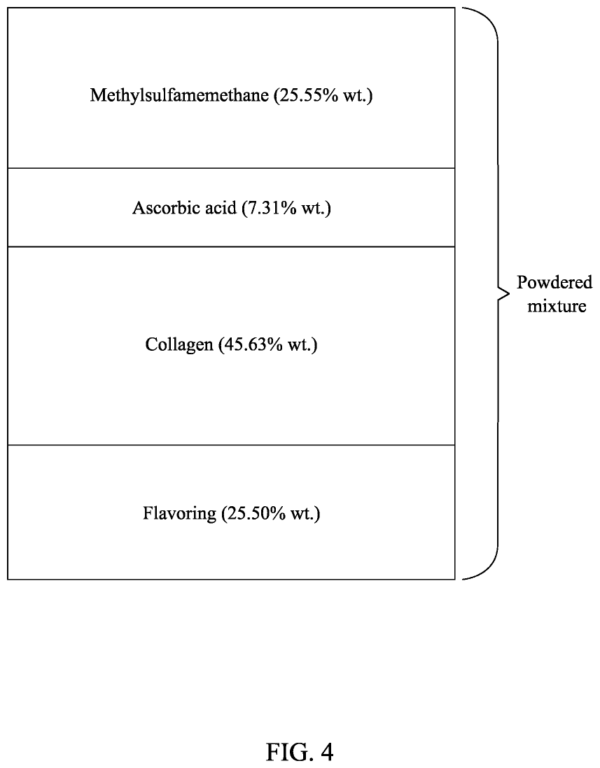

In order to provide the necessary flavor or overcome any residual flavor of the quantity of MSM, the quantity of ascorbic acid, and the quantity of hydrolyzed bovine collagen, the quantity of flavoring is further heterogeneously mixed into the powdered mixture. More specifically, the quantity of flavoring is approximately 21.50 wt. % of the powdered mixture, as seen in FIG. 4. In order to maintain the health benefits of the quantity of MSM, the quantity of ascorbic acid, and the quantity of hydrolyzed bovine collagen, with the addition of the quantity of flavoring, the quantity of MSM is approximately 25.55 wt. % of the powdered mixture. Furthermore, the quantity of ascorbic acid is approximately 7.31 wt. % of the powdered mixture. Furthermore, the quantity of hydrolyzed bovine collagen is approximately 45.63 wt. % of the powdered mixture. In reference to the aforementioned compositional proportions, the term “approximately” preferably means within an error range of up to ±0.05 wt. %.

In order for the present invention to fully effect the body of a user and be digested by the user, the present invention may further comprise a quantity of solvent. The quantity of solvent is preferably a neutral tasting drink or beverage that serves to evenly dilute the powdered mixture. Furthermore, the quantity of powdered mixture is a quantity of solute. In order to create a smooth and appetizing beverage for the user, the quantity of solute is homogeneously mixed into the quantity of solvent to form a collagen-based drink beverage. The collagen-based drink is now a daily supplement that a user consumes in order to improve various body parts while simultaneously slowing down the aging process for the skin. In the preferred embodiment of the present invention, the quantity of solvent is water, and the quantity of solvent may either be hot or cold. However, in various embodiments of the present invention, the quantity of solvent may be a flavored beverage. In order to thoroughly mix the quantity of solute, the quantity of solute is approximately 13.8 grams, and the quantity of solvent is approximately 12 to 16 ounces. Although the invention has been explained in relation to its preferred embodiment, it is to be understood that many other possible modifications and variations can be made without departing from the spirit and scope of the invention as hereinafter claimed.

What is claimed is:

1. A composition for collagen-based drink mix comprising:
a quantity of methylsulfamemethane (MSM);
a quantity of ascorbic acid;
a quantity of hydrolyzed bovine collagen; and
the quantity of MSM, the quantity of ascorbic acid, and the quantity of hydrolyzed bovine collagen being heterogeneously mixed into a powdered mixture.

2. The composition for collagen-based drink mix claimed in claim 1 comprising:
the quantity of MSM being approximately 32.55 percentage by weight (wt. %) of the powdered mixture.

3. The composition for collagen-based drink mix claimed in claim 1 comprising:
the quantity of ascorbic acid being approximately 9.31 wt. % of the powdered mixture.

4. The composition for collagen-based drink mix claimed in claim 1 comprising:
the quantity of hydrolyzed bovine collagen being approximately 58.13 wt. % of the powdered mixture.

5. The composition for collagen-based drink mix claimed in claim 1 comprising:
a quantity of flavoring; and
the quantity of flavoring being further heterogeneously mixed into the powdered mixture.

6. The composition for collagen-based drink mix claimed in claim 5 comprising:
the quantity of flavoring being approximately 21.50 wt. % of the powdered mixture.

7. The composition for collagen-based drink mix claimed in claim 5 comprising:
the quantity of MSM being approximately 25.55 wt. % of the powdered mixture.

8. The composition for collagen-based drink mix claimed in claim 5 comprising:
the quantity of ascorbic acid being approximately 7.31 wt. % of the powdered mixture.

9. The composition for collagen-based drink mix claimed in claim 5 comprising:
the quantity of hydrolyzed bovine collagen being approximately 45.63 wt. % of the powdered mixture.

10. The composition for hill-based protein supplement claimed in claim 5 comprising:
the quantity of flavoring being at least one flavoring ingredient selected from a group consisting of: a quantity of citric acid, a quantity of silicon dioxide, a quantity of malic acid, a quantity of acesulfame potassium, a quantity of sucralose, and a combination thereof.

11. A composition for collagen-based drink mix comprising:
a quantity of methylsulfamemethane (MSM);
a quantity of ascorbic acid;
a quantity of hydrolyzed bovine collagen;
the quantity of MSM, the quantity of ascorbic acid, and the quantity of hydrolyzed bovine collagen being heterogeneously mixed into a powdered mixture;
the quantity of MSM being approximately 32.55 percentage by weight (wt. %) of the powdered mixture;
the quantity of ascorbic acid being approximately 9.31 wt. % of the powdered mixture; and
the quantity of hydrolyzed bovine collagen being approximately 58.13 wt. % of the powdered mixture.

12. A composition for collagen-based drink mix comprising:
a quantity of methylsulfamemethane (MSM);
a quantity of ascorbic acid;
a quantity of hydrolyzed bovine collagen;
a quantity of flavoring; and
the quantity of MSM, the quantity of ascorbic acid, the quantity of hydrolyzed bovine collagen, and the quantity of flavoring being heterogeneously mixed into a powdered mixture.

13. The composition for collagen-based drink mix claimed in claim 12 comprising:
the quantity of flavoring being approximately 21.50 wt. % of the powdered mixture.

14. The composition for collagen-based drink mix claimed in claim 12 comprising:
the quantity of MSM being approximately 25.55 wt. % of the powdered mixture.

15. The composition for collagen-based drink mix claimed in claim 12 comprising:
the quantity of ascorbic acid being approximately 7.31 wt. % of the powdered mixture.

16. The composition for collagen-based drink mix claimed in claim 12 comprising:
the quantity of hydrolyzed bovine collagen being approximately 45.63 wt. % of the powdered mixture.

17. The composition for hill-based protein supplement claimed in claim 12 comprising:
the quantity of flavoring being at least one flavoring ingredient selected from a group consisting of: a quantity of citric acid, a quantity of silicon dioxide, a quantity of malic acid, a quantity of acesulfame potassium, a quantity of sucralose, and a combination thereof.
